(19) United States
(12) Patent Application Publication
(10) Pub. No.: US 2025/0223609 A1
(43) Pub. Date: Jul. 10, 2025

(54) REDUCTION OF IRON LEVELS BY IRON RESPONSIVE PROTEIN SEQUESTRATION WITH SHORT RNAS

(71) Applicant: CORNELL UNIVERSITY, Ithaca, NY (US)

(72) Inventors: Samie R. JAFFREY, New York, NY (US); Jacob L. LITKE, New York, NY (US)

(21) Appl. No.: 18/853,779

(22) PCT Filed: Apr. 4, 2023
(86) PCT No.: PCT/US23/65330

§ 371 Date: Oct. 3, 2024

Related U.S. Application Data
(60) Provisional application No. 63/327,099, filed on Apr. 4, 2022.

(51) Int. Cl. C12N 15/85; C07K 14/47

(52) U.S. Cl. CPC C12N 15/85; C07K 14/4702; C12N 2830/002; C12N 2840/002

(57) ABSTRACT

The present disclosure relates to a circular RNA molecule having an iron responsive element (IRE) comprising the sequence of SEQ ID NO: 1, SEQ ID NO:4, or a portion thereof sufficient to allow for binding to an iron responsive protein. Also disclosed are DNA constructs encoding the circular RNA molecule, host cells that include the circular RNA molecule, pharmaceutical compositions comprising the circular RNA molecule or DNA constructs encoding the circular RNA molecule, and methods of treating a disease or disorder mediated by iron overload.

This application claims the priority benefit of U.S. Provisional Patent Application Ser. No. 63/327,099, filed Apr. 4, 2022, which is hereby incorporated by reference in its entirety.

This invention was made with government support under grant number R01 NS064516 awarded by the National Institutes of Health. The government has certain rights in the invention.

### FIELD

The present invention relates to nucleic acid molecules that bind specifically to iron regulatory proteins, vectors encoding such nucleic acid molecules, and their use in the treatment of a disease or disorder mediated by elevated intracellular iron levels.

### BACKGROUND

Ferroptosis is a form of programmed cell death that depends on iron. It is different from apoptosis in that it uses iron to catalyze a unique form of death.

Ferroptosis has been implicated in diverse neurological diseases, emphysema and metabolic disorders, such as non-alcoholic steatohepatitis and non-alcoholic fatty liver disease. Ferroptosis inhibitors could be useful for treating diseases linked to ferroptosis.

Iron can cause disease in a manner that does not require ferroptosis. High levels of iron are a problem implicated in several diseases. Several diseases associated with elevated iron include: various neurodegenerative diseases, such as Alzheimer's, Huntington's, and Parkinson's diseases, Friedreich's ataxia, traumatic brain injury, and ischemic stroke. Additionally, transfusional iron overload and related conditions such as thalassemia major may be ameliorated with this technology. Ferroptosis may be the disease mechanism, but regardless, iron is thought to contribute to or cause disease pathogenesis.

There are various small molecules of different classes that chelate iron. These chelator molecules sequester iron and depend on export or secretion of the complex. Two major problems with this technology are (1) delivery to relevant tissues and solubility of the chelators and (2) sequestration of iron leads to compensatory regulation of iron metabolism proteins (especially transferrin receptor, ferritin, and ferroportin) that counteract the activity of chelators, leading to increase in total iron levels.

Along with iron chelators, ferrostatin-1 (Fer-1), liproxstatin, and vitamin E inhibit ferroptosis by preventing formation of lipid peroxides. Activation of GPX4 is effective at ferroptosis inhibition. RNAi that targets IRP1 and/or IRP2 could conceivably work; however, IRP1 has a separate distinct function as an aconitase that could be dramatically affected. This method is also not quickly tunable because protein levels do not rapidly recover after downregulation.

The present disclosure is directed to overcoming these and other deficiencies in the art.

### SUMMARY

A first aspect of the disclosure relates to a circular RNA molecule having an iron responsive element (IRE) comprising the sequence of SEQ ID NO:1 or a portion thereof sufficient to allow for binding to an iron responsive protein. In some embodiments, the iron responsive element is an engineered iron responsive element. The iron responsive protein is preferably iron regulatory protein 1 (“IRP1”) and/or iron regulatory protein 2 (“IRP2”).

Another aspect of the present disclosure relates to a circular RNA molecule having an iron responsive element (IRE) comprising the sequence of SEQ ID NO:4 or a portion thereof sufficient to allow for binding to an iron responsive element. In some embodiments, the iron responsive element is an engineered iron responsive element. The iron responsive protein is preferably iron regulatory protein 1 (“IRP1”) and/or iron regulatory protein 2 (“IRP2”).

Another aspect of the disclosure relates to a DNA construct that encodes a circular RNA molecule according to the present disclosure. The DNA construct can be in the form of an isolated transgene or an expression vector (i.e., that include appropriate regulatory sequences to allow for expression of the encoded RNA molecules).

A further aspect of the disclosure relates to a host cell that includes a circular RNA molecule or a DNA construct according to the present disclosure.

Another aspect of the disclosure relates to a pharmaceutical composition comprising (i) a circular RNA molecule or a DNA construct according to the present disclosure and (ii) a pharmaceutically-acceptable carrier.

A further aspect of the disclosure relates to a method of treating a disease or disorder mediated by iron overload. This method involves administering to a subject in need of treatment for a disease or disorder mediated by iron overload a circular RNA molecule, a DNA construct, or a pharmaceutical composition according to the present disclosure, where said administering is effective to treat the disease or disorder mediated by iron overload in the subject.

The present disclosure provides an alternative approach to treat ferroptosis as well as diseases and disorders mediated by iron overload.

### BRIEF DESCRIPTION OF THE DRAWINGS

FIG. 1 is a schematic illustrating how RNA molecules comprising an iron responsive element (IRE) sequester iron responsive protein and trigger a cellular response to high iron levels. In a low iron state, iron responsive protein (IRP) binds to mRNAs such that import of iron by transferrin receptor is increased and storage of iron by ferritin is reduced. IRP regulates mRNA levels by binding to TRE sequences in mRNA. This regulation is reversed in high iron states because IRP binds to either IRE or iron in iron sulfur clusters. Circular RNA molecules comprising an engineered TRE as described herein mimic the higher iron state, even when iron is in fact low. This works by shifting the metabolic set point for iron levels.

FIG. 2 is a schematic illustrating the consensus sequence of SEQ ID NO:1.

FIGS. 3A-3B demonstrate the effects of circular IRE RNA on iron metabolism in cells. FIG. 3A is a Western blot showing the level of transferrin receptor (iron import) and of ferritin (iron storage) in different conditions. FIG. 3B is a bar graph quantifying the changes in iron import and storage.

FIGS. 4A-4B demonstrate the effects of circular IRE RNA on induction of ferroptosis. FIG. 4A are images showing cell viability following the induction of ferroptosis in untransfected HepG2 cells and cells transfected with circular IREs. FIG. 4B shows the amount of lipid peroxidation in HEK293T cells transfected with circular TRE RNAs or control circular RNA after induction of ferroptosis with erastin or FIN56.

### DETAILED DESCRIPTION

Unless otherwise indicated, the definitions and embodiments described in this and other sections are intended to be applicable to all embodiments and aspects of the present application herein described for which they are suitable as would be understood by a person skilled in the art.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “and/or” as used herein means that the listed items are present, or used, individually or in combination. In effect, this term means that “at least one of” or “one or more” of the listed items is used or present.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of.” The methods, kits, systems, and/or compositions of the present disclosure can comprise, consist essentially of, or consist of, the components disclosed.

In embodiments comprising an “additional” or “second” component, the second component as used herein is different from the other components or first component. A “third” component is different from the other, first, and second components, and further enumerated or “additional” components are similarly different.

The term “complementary” when used in connection with nucleic acid, refers to the pairing of bases, A with T or U, and G with C. The term “complementary” refers to nucleic acid molecules that are completely complementary, that is, form A to T or U pairs and G to C pairs across the entire reference sequence, as well as molecules that are partially (e.g., at least 80%, 81%, 82%, 83%, 84%, 85%, 86%, 87%, 88%, 89%0, 90%, 91%, 92%, 93%, 94%, 95%, 96%, 97%, 98%, or 99%) complementary.

The terms “nucleic acid” and “nucleotide” encompass both DNA and RNA unless specified otherwise.

The term “polypeptide,” “peptide”, or “protein” are used interchangeably and to refer to a polymer of amino acid residues. The terms encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “express” and “expression” mean allowing or causing the information in a DNA sequence to become produced, for example producing an RNA by activating the cellular functions involved in transcription of a DNA sequence.

As used herein, the “DNA constructs” of the disclosure are nucleic acid molecules containing a combination of two or more genetic elements not naturally occurring together. Each DNA construct comprises a non-naturally occurring nucleotide sequence that can be in the form of linear DNA or circular DNA, i.e., placed within a vector.

Certain terms employed in the specification, examples, and claims are collected herein. Unless defined otherwise, all technical and scientific terms used in this disclosure have the same meanings as commonly understood by one of ordinary skill in the art to which this disclosure belongs.

Preferences and options for a given aspect, feature, embodiment, or parameter of the disclosure should, unless the context indicates otherwise, be regarded as having been disclosed in combination with any and all preferences and options for all other aspects, features, embodiments, and parameters of the disclosure.

### Circular RNA Molecules

The present disclosure relates to nucleic acid molecules that bind specifically to iron regulatory proteins, vectors encoding such nucleic acid molecules, and their use in the treatment of a disease or disorder mediated by elevated intracellular iron levels.

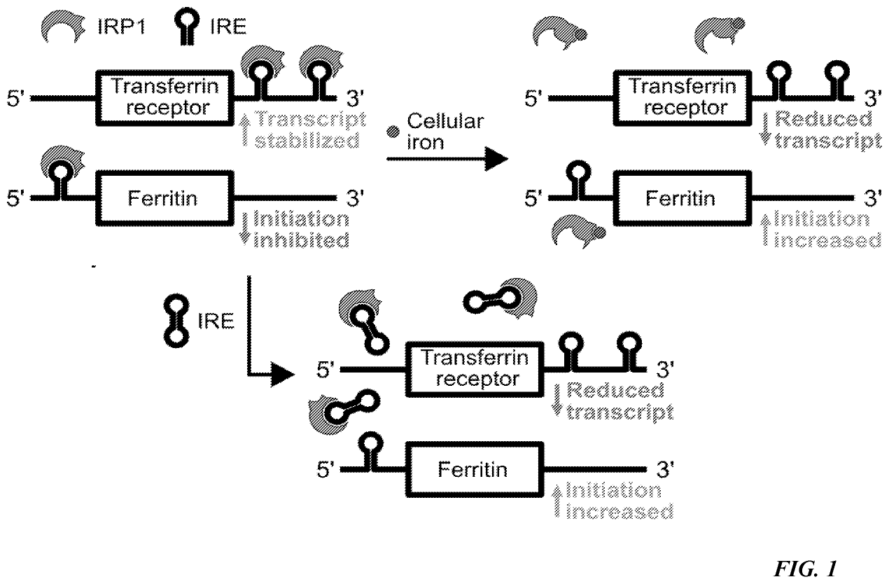

As illustrated in FIG. 1, in a low iron state, IRP binds to mRNAs such that import of iron by transferrin receptor is increased and storage of iron by ferritin is reduced. IRP regulates mRNA levels by binding to IRE sequences in mRNA. This regulation is reversed in high iron states because IRP binds to either IRE or iron in iron sulfur clusters.

Without being bound by theory, short RNAs containing the IRE sequence (e.g., a circular RNA) are also able to mimic the higher iron state, even when iron is in fact low. This works by shifting the metabolic set point for iron levels.

Accordingly, one aspect of the present disclosure relates to a circular RNA molecule having an iron responsive element (IRE) comprising the sequence of SEQ ID NO:1 or a portion thereof sufficient to allow for binding to an iron responsive protein.

Another aspect of the present disclosure relates to a circular RNA molecule having an iron responsive element (IRE) comprising the sequence of SEQ ID NO:4 or a portion thereof sufficient to allow for binding to an iron responsive element.

As described herein, the term “circular RNA” refers to a single stranded, covalently closed loop RNA molecule having no 5′ or 3′ ends.

The iron responsive element (IRE) may be an engineered iron responsive element. As used herein, the term “engineered iron responsive element” refers to an iron responsive element that has been designed to have a specific structure and/or function (e.g., a circular structure).

The circular RNA molecule may be synthesized (e.g., by chemical synthesis) or in vitro transcribed (e.g., from a Tornado vector) (see, e.g., Litke and Jaffrey, “Highly Efficient Expression of Circular RNA Aptamers in Cells using Autocatalytic Transcripts,” Nat. Biotechnol. 37(6):667-675 (2019) and U.S. Patent Application Publication No. 2021/0340542 to Jaffrey et al., which are hereby incorporated by reference in their entirety). Circular RNA may then be purified by standard methods. As discussed in more detail infra, the purified circular RNA may then be administered to a person or cell, e.g., for treatment purposes.

Prior studies have identified a consensus iron responsive element sequence based on ferritin and transferrin receptor (TfR) mRNAs (see, e.g., Jaffrey et al., “The Interaction between the Iron-Responsive Element Binding Protein and its Cognate RNA is Highly Dependent upon both RNA Sequence and Structure,” Nucleic Acids Res. 21(19):4627-4631 (1993), which is hereby incorporated by reference in its entirety).

The circular RNA molecule may comprise the consensus sequence of SEQ ID NO:1 as follows:

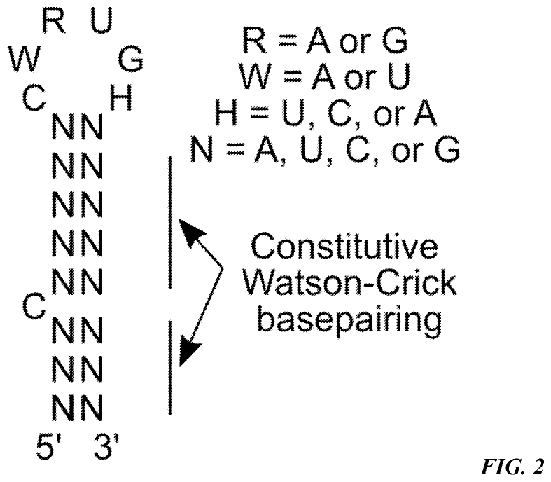

NNNCNNNNNC WRUGHNNNNN NNN,

where N at positions 1, 2, 3, 5, 6, 7, 8, 9, 16, 17, 18, 19, 20, 21, 22, and 23 can be A, U, C, or G; N at positions 1 and 24 form Watson-Crick base pairs; N at positions 2 and 23 form Watson-Crick base pairs; N at positions 3 and 22 form Watson-Crick base pairs; N at positions 5 and 21 form Watson-Crick base pairs; N at positions 6 and 20 form Watson-Crick base pairs; N at positions 7 and 19 form Watson-Crick base pairs; N at positions 8 and 17 form Watson-Crick base pairs; N at positions 9 and 16 form Watson-Crick base pairs; W at position 11 can be A or U; R at position 12 can be A or G; H at position 15 can be U, C, or A. Suitable Watson-Crick base pairs include, e.g., such as G-C(C-G) or A-U (U-A) (FIG. 2). Non-canonical Watson-Crick base pairs between G-U (U-G) are also suitable. Also encompassed are synthetic RNA which may comprise non-natural base pairs designed to maintain the overall helical structure.

An exemplary IRE sequence is SEQ ID NO:3, as follows:

UAUCGGGAGC AGUGUCUUCC AUA.

The circular RNA molecule may comprise the consensus sequence of SEQ ID NO:4 as follows:

NNN UGC NNNNNC WRUGHNNNNN C NNN,

where N at positions 1, 2, 3, 7, 8, 9, 10, 11; 18, 19, 20, 21, 22, 24, 25, and 26 can be A, U, C, or G; W at position 13 can be A or U; R at position 14 can be A or G; H at position 17 can be U, C, or A; N at positions 7 and 22 form Watson-Crick base pairs; N at positions 8 and 21 form Watson-Crick base pairs; N at positions 9 and 20 form Watson-Crick base pairs; N at positions 10 and 19 form Watson-Crick base pairs; N at positions 11 and 18 form Watson-Crick base pairs; e.g., such as G-C(C-G) or A-U (U-A) (FIG. 2). Non-canonical Watson-Crick base pairs between G-U (U-G) are also suitable. Also encompassed are synthetic RNA which may comprise non-natural base pairs designed to maintain the overall helical structure.

An exemplary IRE sequence is SEQ ID NO:5 (Ferritin IRE), as follows: UCU UGC UUCAAC AGUGUUUGAA C GGA (see, e.g., Ke et al., “Loops and Bulge/Loops in Iron-Responsive Element Isoforms Influence Iron Regulatory Protein Binding. Fine-Tuning of mRNA Regulation,” JCB 273(37):P23637-23640 (1998), which is hereby incorporated by reference in its entirety). Additional exemplary IRE sequences including variants thereof are disclosed in, e.g., Ke et al., “Loops and Bulge/Loops in Iron-Responsive Element Isoforms Influence Iron Regulatory Protein Binding. Fine-Tuning of mRNA Regulation,” JCB 273(37):P23637-23640 (1998), which is hereby incorporated by reference in its entirety.

As described supra, iron regulatory proteins (“IRPs”) regulate the expression of genes involved in iron metabolism by binding to RNA stem-loop structures known as iron responsive elements (IREs) in target mRNAs (Zhang et al., “The Physiological Functions of Iron Regulatory Proteins in Iron Homeostasis—An Update,” Frot. Pharmacol. 5:124 (2014), which is hereby incorporated by reference in its entirety).

There are two members of the mammalian IRP protein family, iron regulatory protein 1 (“IRP1”) and iron regulatory protein 2 (“IRP2”). During iron scarcity or oxidative stress, IRP1 binds to mRNA iron responsive elements to modulate the translation of iron metabolism genes. In iron-rich conditions, IRP1 binds an iron-sulfur cluster to function as a cytosolic aconitase (see, e.g., Volz, K., “The Functional Duality of Iron Regulatory Protein 1,” Curr. Opin. Struct. Biol. 18(1):106-111(2008), which is hereby incorporated by reference in its entirety). The amino acid sequence homology is high between IRP1 and IRP2 throughout the entire length of the protein, with the exception of a unique insertion of 73 amino acids in IPR2 relative to IPR1 (see, e.g., Iwai et al., “Requirements for Iron-Regulated Degradation of the RNA Binding Protein, Iron Regulatory Protein 2,” EMBO J. 14(21):5350-5357 (1995), which is hereby incorporated by reference in its entirety). However, the activity of IRP2 is primarily regulated by protein stability. With limiting iron or oxygen, IRP2 is stable and binds IREs (see, e.g., Wang et al., “FBXL5 Regulated IRP2 Stability in Iron Homeostasis via and Oxygen-Responsive [2Fe2S] Cluster,” Molecular Cell 78(1):31-45 (2020), which is hereby incorporated by reference in its entirety). Under iron- and oxygen-enriched conditions, IRP2 undergoes ubiquitin-dependent proteasomal degradation (see, e.g., Guo et al., “Iron Regulates the Intracellular Degradation of Iron Regulatory Protein 2 by the Proteasome,” J. Biol. Chem. 270(37):21645-21651(1995) and Iwai et al., “Requirements for Iron-Regulated Degradation of the RNA Binding Protein, Iron Regulatory Protein 2,” EMBO J. 14(21):5350-5357 (1995), which are hereby incorporated by reference in their entirety).

In some embodiments, the iron responsive protein is iron regulatory protein 1 (“IRP1”) and/or iron regulatory protein 2 (“IRP2”).

The circular RNA molecule may comprise a non-natural or modified nucleotide. In some embodiments, the RNA molecule comprises a modified base.

As used herein, a “modified base” is, according to one embodiment, a ribonucleotide base of uracil, cytosine, adenine, or guanine that possesses a chemical modification from its normal structure. For example, one type of modified base is a methylated base, such as N⁶-methyladenosine (m⁶A). A modified base may also be a substituted base, meaning the base possesses a structural modification that renders it a chemical entity other than uracil, cytosine, adenine, or guanine. For example, pseudouridine is one type of substituted RNA base. Table 1 below provides a list of modified bases that may be present in the RNA molecule of the present invention.

TABLE 1
List of Base Modifications

| Abbreviation | Chemical name |
|---|---|
| m¹acp³Y | 1-methyl-3-(3-amino-3-carboxypropyl) pseudouridine |
| m¹A | 1-methyladenosine |
| m¹G | 1-methylguanosine |
| m¹I | 1-methylinosine |
| m¹Y | 1-methylpseudouridine |
| m¹Am | 1,2′-O-dimethyladenosine |
| m¹Gm | 1,2′-O-dimethylguanosine |
| m¹Im | 1,2′-O-dimethylinosine |
| m²A | 2-methyladenosine |
| ms²io⁶A | 2-methylthio-N⁶-(cis-hydroxyisopentenyl) adenosine |
| ms²hn⁶A | 2-methylthio-N⁶-hydroxynorvalyl carbamoyladenosine |
| ms²i⁶A | 2-methylthio-N⁶-isopentenyladenosine |
| ms²m⁶A | 2-methylthio-N⁶-methyladenosine |
| ms²t⁶A | 2-methylthio-N⁶-threonyl carbamoyladenosine |
| s²Um | 2-thio-2′-O-methyluridine |
| s²C | 2-thiocytidine |
| s²U | 2-thiouridine |
| Am | 2′-O-methyladenosine |
| Cm | 2′-O-methylcytidine |
| Gm | 2′-O-methylguanosine |
| Im | 2′-O-methylinosine |
| Ym | 2′-O-methylpseudouridine |
| Um | 2′-O-methyluridine |
| Ar(p) | 2′-O-ribosyladenosine (phosphate) |
| Gr(p) | 2′-O-ribosylguanosine (phosphate) |
| acp³U | 3-(3-amino-3-carboxypropyl)uridine |
| m³C | 3-methylcytidine |
| m³Y | 3-methylpseudouridine |
| m³U | 3-methyluridine |
| m³Um | 3,2′-O-dimethyluridine |
| imG-14 | 4-demethylwyosine |
| s⁴U | 4-thiouridine |
| chm⁵U | 5-(carboxyhydroxymethyl)uridine |
| mchm⁵U | 5-(carboxyhydroxymethyl)uridine methyl ester |
| inm⁵s²U | 5-(isopentenylaminomethyl)-2-thiouridine |
| inm⁵Um | 5-(isopentenylaminomethyl)-2′-O-methyluridine |
| inm⁵U | 5-(isopentenylaminomethyl)uridine |
| nm⁵s²U | 5-aminomethyl-2-thiouridine |
| ncm⁵Um | 5-carbamoylmethyl-2′-O-methyluridine |
| ncm⁵U | 5-carbamoylmethyluridine |
| cmnm⁵Um | 5-carboxymethylaminomethyl-2′-O-methyluridine |
| cmnm⁵s²U | 5-carboxymethylaminomethyl-2-thiouridine |
| cmnm⁵U | 5-carboxymethylaminomethyluridine |
| cm⁵U | 5-carboxymethyluridine |
| f⁵Cm | 5-formyl-2′-O-methylcytidine |
| f⁵C | 5-formylcytidine |
| hm⁵C | 5-hydroxymethylcytidine |
| ho⁵U | 5-hydroxyuridine |
| mcm⁵s²U | 5-methoxycarbonylmethyl-2-thiouridine |
| mcm⁵Um | 5-methoxycarbonylmethyl-2′-O-methyluridine |
| mcm⁵U | 5-methoxycarbonylmethyluridine |
| mo⁵U | 5-methoxyuridine |
| m⁵s²U | 5-methyl-2-thiouridine |
| mnm⁵se²U | 5-methylaminomethyl-2-selenouridine |
| mnm⁵s²U | 5-methylaminomethyl-2-thiouridine |
| mnm⁵U | 5-methylaminomethyluridine |
| m⁵C | 5-methylcytidine |
| m⁵D | 5-methyldihydrouridine |
| m⁵U | 5-methyluridine |
| tm⁵s²U | 5-taurinomethyl-2-thiouridine |
| tm⁵U | 5-taurinomethyluridine |
| m⁵Cm | 5,2′-O-dimethylcytidine |
| m⁵Um | 5,2′-O-dimethyluridine |
| preQ₁ | 7-aminomethyl-7-deazaguanosine |
| preQ₀ | 7-cyano-7-deazaguanosine |
| m⁷G | 7-methylguanosine |
| G⁺ | archaeosine |
| D | dihydrouridine |
| oQ | epoxyqueuosine |
| galQ | galactosyl-queuosine |
| OHyW | hydroxywybutosine |
| I | inosine |
| imG2 | isowyosine |
| k²C | lysidine |
| manQ | mannosyl-queuosine |
| mimG | methylwyosine |
| m²G | N²-methylguanosine |
| m²Gm | N²,2′-O-dimethylguanosine |
| m^{2, 7}G | N²,7-dimethylguanosine |
| m^{2, 7}Gm | N²,7,2′-O-trimethylguanosine |
| m²₂G | N²,N²-dimethylguanosine |
| m²₂Gm | N²,N²,2′-O-trimethylguanosine |
| m^{2, 2, 7}G | N²,N²,7-trimethylguanosine |
| ac⁴Cm | N⁴-acetyl-2′-O-methylcytidine |
| ac⁴C | N⁶-acetylcytidine |
| m⁴C | N⁴-methylcytidine |
| m⁴Cm | N⁴,2′-O-dimethylcytidine |
| m⁴₂Cm | N⁴,N⁴,2′-O-trimethylcytidine |
| io⁶A | N⁴-(cis-hydroxyisopentenyl)adenosine |
| ac⁶A | N⁶-acetyladenosine |
| g⁶A | N⁶-glycinylcarbamoyladenosine |
| hn⁶A | N⁶-hydroxynorvalylcarbamoyladenosine |
| i⁶A | N⁶-isopentenyladenosine |
| m⁶t⁶A | N⁶-methyl-N⁶-threonylcarbamoyladenosine |
| m⁶A | N⁶-methyladenosine |
| t⁶A | N⁶-threonylcarbamoyladenosine |
| m⁶Am | N⁶,2′-O-dimethyladenosine |
| m⁶₂A | N⁶,N⁶-dimethyladenosine |
| m⁶₂Am | N⁶,N⁶,2′-O-trimethyladenosine |
| o₂yW | peroxywybutosine |
| Y | pseudouridine |
| Q | queuosine |
| OHyW | undermodified hydroxywybutosine |
| cmo⁵U | uridine 5-oxyacetic acid |
| mcmo⁵U | uridine 5-oxyacetic acid methyl ester |
| yW | wybutosine |
| imG | wyosine |

The circular RNA molecules of the present disclosure also include PNAs and phosphorthioate or nucleic acids with other backbones.

The circular RNA molecule may further comprise an RNA scaffold. According to some embodiments, the RNA scaffold is an F29 or F30 scaffold (see, e.g., Filonov et al., “In-Gel Imaging of RNA Processing Using Broccoli Reveals Optimal Aptamer Expression Strategies,” Chem. Biol. 22(5): 649-660 (2015), which is hereby incorporated by reference in its entirety).

The circular RNA molecule may further comprise a fluorogenic aptamer. Fluorogenic aptamers are well known in the art and include, without limitation, Squash, Beetroot, Spinach, Spinach 2, Broccoli, Red-Broccoli, Orange Broccoli, Corn, Mango, Malachite Green, cobalamine-binding aptamer, and derivatives thereof. See, e.g., Truong et al., “The Fluorescent Aptamer Squash Extensively Repurposes the Adenine Riboswitch Fold, Nat. Chem. Biol. 18(2):191-198 (2022); Wu et al., “Self-Assembly of Intracellular Multivalent RNA Complexes Using Dimeric Corn and Beetroot Aptamers,” J. Am. Chem. Soc. (2022); Autour et al., “Fluorogenic RNA Mango Aptamers for Imaging Small Non-Coding RNAs in Mammalian Cells,” Nature Comm. 9:Article 656 (2018); Jaffrey, S., “RNA-Based Fluorescent Biosensors for Detecting Metabolites In Vitro and in Living Cells,” Adv Pharmacol. 82:187-203 (2018); and Litke et al., “Developing Fluorogenic Riboswitches for Imaging Metabolite Concentration Dynamics in Bacterial Cells,” Methods Enzymol. 572:315-33 (2016), each of which are hereby incorporated by reference in their entirety). In some embodiments, the fluorogenic aptamer binds to a fluorophore whose fluorescence, absorbance, spectral properties, or quenching properties are increased, decreased, or altered by interaction with the fluorogenic aptamer. Any aptamer-dye complex, some of which are fluorogenic aptamers, may be used. In addition, some aptamers can bind quenchers and some do other things to change the photophysical properties of dyes.

The fluorogenic aptamer may bind to a fluorophore whose fluorescence, absorbance, spectral properties, or quenching properties are increased, decreased, or altered by interaction with the fluorogenic aptamer.

### DNA Constructs

The circular RNA molecules of the present disclosure can be prepared either in vitro or in vivo using recombinant constructs, including transgenes, that encode the circular RNA molecules of the present disclosure. Whether using in vitro transcription or transgenes suitable for expression in vivo, these genetic constructs can be prepared using well known recombinant techniques.

Another aspect of the disclosure relates to a DNA construct that encodes a circular RNA molecule according to the present disclosure. The DNA construct can be in the form of an isolated transgene or an expression vector (i.e., that include appropriate regulatory sequences to allow for expression of the encoded RNA molecules).

According to some embodiments, the DNA construct comprises a nucleic acid sequence encoding: (i) a first self-cleaving ribozyme; (ii) a first ligation sequence; (iii) the iron responsive element of SEQ ID NO:1 or SEQ ID NO:4; (iv) a second ligation sequence; and (v) a second self-cleaving ribozyme.

In some embodiments, the DNA construct comprises a nucleic acid sequence encoding: (i) a first self-cleaving ribozyme; (ii) a first ligation sequence; (iii) the iron responsive element of SEQ ID NO:1; (iv) a second ligation sequence; and (v) a second self-cleaving ribozyme.

In some embodiments, the DNA construct comprises a nucleic acid sequence encoding: (i) a first self-cleaving ribozyme; (ii) a first ligation sequence; (iii) the iron responsive element of SEQ ID NO:4; (iv) a second ligation sequence; and (v) a second self-cleaving ribozyme.

As used herein, the term “ribozyme” refers to an RNA sequence that hybridizes to a complementary sequence in a substrate RNA and cleaves the substrate RNA in a sequence specific manner at a substrate cleavage site. Typically, a ribozyme contains a catalytic region flanked by two binding regions. The ribozyme binding regions hybridize to the substrate RNA, while the catalytic region cleaves the substrate RNA at a substrate cleavage site to yield a cleaved RNA product. The nucleotide sequence of the ribozyme binding regions may be completely complementary or partially complementary to the substrate RNA sequence with which the ribozyme hybridizes.

In some embodiments, each of the first ribozyme and the second ribozyme comprise a sequence that may be cleaved to produce a 5′-OH end and a 2′,3′-cyclic phosphate end.

Self-cleaving ribozymes are characterized by distinct active site architectures and divergent, but similar, biochemical properties. The cleavage activities of self-cleaving ribozymes are highly dependent upon divalent cations, pH, and base-specific mutations, which can cause changes in the nucleotide arrangement and/or electrostatic potential around the cleavage site (see, e.g., Weinberg et al., “New Classes of Self-Cleaving Ribozymes Revealed by Comparative Genomics Analysis,” Nat. Chem. Biol. 11(8): 606-610 (2015) and Lee et al., “Structural and Biochemical Properties of Novel Self-Cleaving Ribozymes,” Molecules 22(4):E678 (2017), which are hereby incorporated by reference in their entirety).

Suitable self-cleaving ribozymes include, but are not limited to, Hammerhead, Hairpin, Hepatitis Delta Virus (“HDV”), Neurospora Varkud Satellite (“VS”), Vg1, glucosamine-6-phosphate synthase (glmS), Twister, Twister Sister, Hatchet, Pistol, and engineered synthetic ribozymes, and derivatives thereof (see, e.g., Harris et al., “Biochemical Analysis of Pistol Self-Cleaving Ribozymes,” RNA 21(11):1852-8 (2015), which is hereby incorporated by reference in its entirety).

Twister ribozymes comprise three essential stems (P1, P2, and P4), with up to three additional ones (P0, P3, and P5) of optional occurrence. Three different types of Twister ribozymes have been identified depending on whether the termini are located within stem P1 (type P1), stem P3 (type P3), or stem P5 (type P5) (see, e.g., Roth et al., “A Widespread Self-Cleaving Ribozyme Class is Revealed by Bioinformatics,” Nature Chem. Biol. 10(1):56-60 (2014)). The fold of the Twister ribozyme is predicted to comprise two pseudoknots (T1 and T2, respectively), formed by two long-range tertiary interactions (see Gebetsberger et al., “Unwinding the Twister Ribozyme: from Structure to Mechanism,” WIREs RNA 8(3):e1402 (2017), which is hereby incorporated by reference in its entirety).

Twister Sister ribozymes are similar in sequence and secondary structure to Twister ribozymes. In particular, some Twister RNAs have P1 through P5 stems in an arrangement similar to Twister Sister and similarities in the nucleotides in the P4 terminal loop exist. However, these two ribozyme classes cleave at different sites, Twister Sister ribozymes do not appear to form pseudoknots via Watson-Crick base pairing (which occurs in all known twister ribozymes), and there is poor correspondence among many of the most highly conserved nucleotides in each of these two motifs (see Weinberg et al., “New Classes of Self-Cleaving Ribozymes Revealed by Comparative Genomics Analysis,” Nat. Chem. Biol. 11(8):606-610 (2015), which is hereby incorporated by reference in its entirety).

Pistol ribozymes are characterized by three stems: P1, P2, and P3, as well as a hairpin and internal loops. A six-base-pair pseudoknot helix is formed by two complementary regions located on the P1 loop and the junction connecting P2 and P3; the pseudoknot duplex is spatially situated between stems P1 and P3 (Lee et al., “Structural and Biochemical Properties of Novel Self-Cleaving Ribozymes,” Molecules 22(4):E678 (2017), which is hereby incorporated by reference in its entirety).

Hammerhead ribozymes are composed of structural elements including three helices, referred to as stem I, stem II, and stem III, and joined at a central core of 11-12 single strand nucleotides. Hammerhead ribozymes may also contain loop structures extending from some or all of the helices. These loops are numbered according to the stem from which they extend (e.g., loop I, loop II, and loop III).

In some embodiments, the first ribozyme is a Twister ribozyme or a Twister Sister ribozyme. For example, the first ribozyme may be a P3 Twister ribozyme.

In some embodiments, the second ribozyme is a Twister, Twister Sister, or Pistol Ribozyme. For example, the second ribozyme may be a P1 Twister ribozyme.

In some embodiments, the first ribozyme is a P3 Twister ribozyme and the second ribozyme is a P1 Twister ribozyme.

The ribozymes of the present disclosure include naturally-occurring (wildtype) ribozymes and modified ribozymes, e.g., ribozymes containing one or more modifications, which can be addition, deletion, substitution, and/or alteration of at least one (or more) nucleotide. Such modifications may result in the addition of structural elements (e.g., a loop or stem), lengthening or shortening of an existing stem or loop, changes in the composition or structure of a loop(s) or a stem(s), or any combination of these. As described herein, modification of the nucleotide sequence of naturally occurring self-cleaving ribozymes (e.g., a P3 Twister ribozyme) can increase or decrease the ability of a ribozyme to autocatalytically cleave its RNA. In some embodiments, each of the first and the second ribozyme is, independently, modified to comprise a non-natural or modified nucleotide. In some embodiments, each of the first and the second ribozyme is modified to comprise pseudouridine in place of uridine.

In some embodiments, each of the first and the second ribozyme is, independently, a split ribozyme or ligand-activated ribozyme derivative.

As used herein, the phrase “ligation sequence” refers to a sequence complementary to another sequence, which enables the formation of Watson-Crick base pairing to form suitable substrates for ligation by a ligase, e.g., an RNA ligase. In some embodiments, each of the first ligation sequence and the second ligation sequence comprise a portion of a tRNA exon sequence or derivative thereof. The first ligation sequence and the second ligation sequence may each, independently, comprise at least 1, 2, 3, 4, 5, 6, 7, 8, 9, 10, 11, or 12 additional nucleotides to promote base-pairing with each other.

In some embodiments, the first ligation sequence and the second ligation sequence are substrates for an RNA ligase. According to some embodiments, the RNA ligase is RtcB. RtcB is not present in all lower organisms, but molecules with similar activities are present. In other words, there are molecules that ligate ends similar to the ligation activity of RtcB. RtcB (or other functionally similar molecules) may be overexpressed to maximize circular RNA expression according to the present disclosure.

Ligation sequences assist in circularization of the RNA molecule, to protect the RNA molecule from degradation and, therefore, ultimately enhance expression of the effector molecule. While it is thought that the RNA molecule of the present disclosure could circularize without the ligation sequences, and such an invention is hereby contemplated, the ligation sequences are also believed to cause the RNA ends to more efficiently come together for the RNA ligase (e.g., RtcB). In other words, the ligation sequences are believed to help draw proper 5′ and 3′ ends of the RNA molecule closer to each other to assist in the circularization of the RNA molecule.

Suitable iron responsive elements comprising the sequence of SEQ ID NO:1 are described supra.

Transcription of the DNA molecule described herein is dependent upon the presence of a promoter, which is a DNA sequence that directs the binding of RNA polymerase and thereby promotes RNA synthesis. Accordingly, the DNA construct of the present disclosure may include a promoter operably coupled to the nucleic acid sequence encoding the first self-cleaving ribozyme, the first ligation sequence, the TRE comprising the sequence of SEQ ID NO:1, the second ligation sequence, and the second self-cleaving ribozyme to control expression of the nucleic acid sequence.

In some embodiments, the DNA construct of the present disclosure may include a promoter operably coupled to the nucleic acid sequence encoding the first self-cleaving ribozyme, the first ligation sequence, the TRE comprising the sequence of SEQ ID NO:4, the second ligation sequence, and the second self-cleaving ribozyme to control expression of the nucleic acid sequence.

As used herein, the term “promoter” refers to a DNA sequence which contains the binding site for RNA polymerase and initiates transcription of a downstream nucleic acid sequence.

The DNA sequences of eukaryotic promoters differ from those of prokaryotic promoters. Furthermore, eukaryotic promoters and accompanying genetic signals may not be recognized in or may not function in a prokaryotic system and, further, prokaryotic promoters are not recognized and do not function in eukaryotic cells.

Promoters vary in their “strength” (i.e., their ability to promote transcription). It is desirable to use strong promoters to obtain a high level of transcription. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen to inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

As described above, one type of regulatory sequence is a promoter located upstream or 5′ to the coding sequence of the DNA molecule. Depending upon the desired activity, it is possible to select the promoter for not only in vitro production of the nucleic acid sequence encoding the first self-cleaving ribozyme, the first ligation sequence, the TRE comprising the sequence of SEQ ID NO:1 or SEQ ID NO:4, the second ligation sequence, and the second self-cleaving ribozyme according to the present disclosure, but also in vivo production in cultured cells or whole organisms, as described below. Because in vivo production can be regulated genetically, a suitable type of promoter is an inducible promoter which induces transcription of the DNA molecule in response to specific conditions, thereby enabling expression of the nucleic acid sequence encoding the first self-cleaving ribozyme, the first ligation sequence, the TRE comprising the sequence of SEQ ID NO:1 or SEQ ID NO:4, the second ligation sequence, and the second self-cleaving ribozyme as desired (i.e., expression within specific tissues, or at specific temporal and/or developmental stages).

Suitable promoters for use with the DNA construct molecule of the present disclosure include, without limitation, a T7 promoter, a SUP4 tRNA promoter, an RPR1 promoter, a GPD promoter, a GAL1 promoter, an hsp70 promoter, an Mtn promoter, a UAShs promoter, and functional fragments thereof. The T7 promoter is a well-defined, short DNA sequence that can be recognized and utilized by T7 RNA polymerase of the bacteriophage T7. The T7 RNA polymerase can be purified in large scale and is commercially available. The transcription reaction with T7 promoter can be conducted in vitro to produce a large amount of the molecular complex of the present invention (Milligan et al., “Oligoribonucleotide Synthesis Using T7 RNA Polymerase and Synthetic DNA Templates,” Nucleic Acids Res. 15(21):8783-8798 (1987), which is hereby incorporated by reference in its entirety). The SUP4 tRNA promoter and RPR1 promoter are driven by RNA polymerase III of the yeast Saccharomyces cerevisiae, and suitable for high level expression of RNA less than 400 nucleotides in length (Kurjan et al., Mutation at the Yeast SUP4 tRNA^{tyr} Locus: DNA Sequence Changes in Mutants Lacking Suppressor Activity,” Cell 20:701-709 (1980); Lee et al., “Expression of RNase P RNA in Saccharomyces cerevisiae is Controlled by an Unusual RNA Polymerase III Promoter,” Proc. Natl. Acad. Sci. USA 88:6986-6990 (1991), each of which is hereby incorporated by reference in its entirety). The glyceraldehyde-3-phosphate dehydrogenase (GPD) promoter in yeast is a strong constitutive promoter driven by RNA polymerase II (Bitter et al., “Expression of Heterologous Genes in Saccharomyces cerevisiae from Vectors Utilizing the Glyceraldehyde-3-phosphate Dehydrogenase Gene Promoter,” Gene 32:263-274 (1984), which is hereby incorporated by reference in its entirety). The galactokinase (GAL1) promoter in yeast is a highly inducible promoter driven by RNA polymerase II (Johnston and Davis, “Sequences that Regulate the Divergent GAL1-GAL10 Promoter in Saccharomyces cerevisiae,” Mol. Cell. Biol. 4:1440-1448 (1984), which is hereby incorporated by reference in its entirety). The heat shock promoters are heat inducible promoters driven by the RNA polymerase II in eukaryotes. The frequency with which RNA polymerase II transcribes the major heat shock genes can be increased rapidly in minutes over 100-fold upon heat shock. Another inducible promoter driven by RNA polymerase II that can be used in the present invention is a metallothionine (Mtn) promoter, which is inducible to the similar degree as the heat shock promoter in a time course of hours (Stuart et al., “A 12-Base-Pair Motif that is Repeated Several Times in Metallothionine Gene Promoters Confers Metal Regulation to a Heterologous Gene,” Proc. Natl. Acad. Sci. USA 81:7318-7322 (1984), which is hereby incorporated by reference in its entirety).

Initiation of transcription in mammalian cells requires a suitable promoter, which may include, without limitation, β-globin, GAPDH, β-actin, actin, Cstf2t, SV40, MMTV, metallothionine-1, adenovirus Ela, CMV immediate early, immunoglobulin heavy chain promoter and enhancer, and RSV-LTR. Termination of transcription in eukaryotic genes involves cleavage at a specific site in the RNA which may precede termination of transcription. Also, eukaryotic termination varies depending on the RNA polymerase that transcribes the gene. However, selection of suitable 3′ transcription termination regions is well known in the art and can be performed with routine skill.

Spatial control of an RNA molecule can be achieved by tissue-specific promoters, which have to be driven by the RNA polymerase II. The many types of cells in animals and plants are created largely through mechanisms that cause different genes to be transcribed in different cells, and many specialized animal cells can maintain their unique character when grown in culture. The tissue-specific promoters involved in such special gene switching mechanisms, which are driven by RNA polymerase II, can be used to drive the transcription templates that code for the molecular complex of the present invention, providing a means to restrict the expression of the molecular complex in particular tissues. Any of a variety of tissue-specific promoters can be selected as desired.

The promoter according to the present disclosure may be a constitutively active promoter (i.e., a promoter that is constitutively in an active or “on” state), an inducible promoter (i.e., a promoter whose state, active or inactive state, is controlled by an external stimulus, e.g., the presence of a particular temperature, compound, or protein), a spatially restricted promoter (i.e., transcriptional control element, enhancer, etc.) (e.g., tissue specific promoter, cell type specific promoter, etc.), or a temporally restricted promoter (i.e., the promoter is in the “on” state or “off” state during specific stages of a biological process).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., RNA Polymerase I, RNA Polymerase II, RNA Polymerase III). Exemplary promoters include, but are not limited to the SV40 early promoter, mouse mammary tumor virus long terminal repeat (“LTR”) promoter; adenovirus major late promoter (“Ad MLP”); a herpes simplex virus (“HSV”) promoter, a cytomegalovirus (“CMV”) promoter such as the CMV immediate early promoter region (“CMVIE”), a rous sarcoma virus (“RSV”) promoter, a human U6 small nuclear promoter (“U6”) (Miyagishi et al., “U6 promoter-driven siRNAs with four uridine 3′ overhangs efficiently suppress targeted gene expression in mammalian cells,” Nature Biotechnology 20:497-500 (2002), which is hereby incorporated by reference in its entirety), an enhanced U6 promoter (e.g., Xia et al., “An enhanced U6 promoter for synthesis of short hairpin RNA,” Nucleic Acids Res. 31(17):e100 (2003), which is hereby incorporated by reference in its entirety), a human H1 promoter (“H1”), and the like.

Examples of inducible promoters include, but are not limited to, T7 RNA polymerase promoter, T3 RNA polymerase promoter, isopropyl-beta-D-thiogalactopyranoside (IPTG)-regulated promoter, lactose induced promoter, heat shock promoter, tetracycline-regulated promoter, steroid-regulated promoter, metal-regulated promoter, estrogen receptor-regulated promoter, etc. Inducible promoters can therefore be regulated by molecules including, but not limited to, doxycycline, RNA polymerase, e.g., T7 RNA polymerase, an estrogen receptor, an estrogen receptor fusion, etc.

In some embodiments, the promoter is a prokaryotic promoter selected from the group consisting of T7, T3, SP6 RNA polymerases, and derivatives thereof. Additional suitable prokaryotic promoters include, without limitation, T7lac, araBAD, trp, lac, Ptac, and pL promoters.

The promoter may be a eukaryotic RNA Polymerase II promoter or a derivative thereof. Exemplary RNA polymerase II promoters include, without limitation, cytomegalovirus (“CMV”), phosphoglycerate kinase-1 (“PGK-1”), elongation factor 1α (“EF1α”), and ubiquitin C (UbiC) promoters.

In some embodiments, the promoter is a eukaryotic RNA polymerase I promoter, RNA polymerase III promoter, or a derivative thereof. In some embodiments, the promoter is a eukaryotic RNA polymerase III promoter selected from the group consisting of U6, H1, 5S, 7SK, and derivatives thereof.

The RNA Polymerase promoter may be mammalian. Suitable mammalian promoters include, without limitation, human, murine, bovine, canine, feline, ovine, porcine, ursine, and simian promoters. In one embodiment, the RNA polymerase promoter sequence is a human promoter.

In some embodiments, the DNA construct comprises the sequence of SEQ ID NO:2 as follows:

GGCCGCACTCGCCGGTCCCAAGCCCGGATAAAATGGGAGGGGGCG
GGAAACCGCCTaaccatGCCGAGTGCGGCCGCTTGCCATGTGTAT
CGGTCCGCTACAAGGTGAGCCCAATAATACGGTTTGGGTTAGGAT
AGGAAGTAGAGCCGTAAACTCTCTAAGCGGTAGCGGTCCGATACT
CTGaTGATGGGTCCCGATTATCGGGAGCAGTGTCTTCCATAATCG
GGTCCCATCAtTCATGGCAAGTGGCCGCGGTCGGCGTGGACTGTA
GAACACTGCCAATGCCGGTCCCAAGCCCGGATAAAAGTGGAGGGT
ACAGTCCACGC,

where nucleic acid sequences at positions 11-71 correspond to ribozyme 1; nucleic acid sequences at positions 79-90, 176-188, and 232-244 correspond to the F30 scaffold; nucleic acid sequences at positions 98-168 correspond to the fluorophore Squash; nucleic acid sequences at positions 196-224 correspond to an Iron Response Element; and nucleic acid sequences at positions 261-314 correspond to ribozyme 2.

In some embodiments, the DNA construct is an expression vector.

As used herein, the term “vector” refers to any genetic element, such as a plasmid, phage, transposon, cosmid, chromosome, virus, virion, etc., which is capable of replication when associated with the proper control elements and which is capable of transferring gene sequences between cells. Thus, the term includes cloning and expression vectors, as well as viral vectors. The vector contains the necessary elements for the transcription and/or translation of the nucleic acid sequence encoding the effector molecule(s) of the present invention.

In some embodiments, the vector is a plasmid. Numerous vectors suitable for use in the compositions of the present invention are known to those of skill in the art, and many are commercially available. The following vectors are provided by way of example; for eukaryotic cells: pXT1, pSG5 (Stratagene), pSVK3, pBPV, pMSG, and pSVLSV40 (Pharmacia). However, any other vector may be used so long as it is compatible with the cell.

In some embodiments, the vector is a viral vector. Suitable viral expression vectors include, but are not limited to, viral vectors based on vaccinia virus; poliovirus; adenovirus (see, e.g., PCT Publication Nos. WO 94/12649 to Gregory et al., WO 93/03769 to Crystal et al., WO 93/19191 to Haddada et al., WO 94/28938 to Wilson et al., WO 95/11984 to Gregory, and WO 95/00655 to Graham, which are hereby incorporated by reference in their entirety); adeno-associated virus (see, e.g., Ali et al., Hum. Gene Ther. 9:8186 (1998), Flannery et al., PNAS 94:6916-6921 (1997); Bennett et al., Invest. Opthalmol. Vis. Sci. 38:2857-2863 (1997); Jomary et al., Gene Ther. 4:683-690 (1997), Rolling et al., Hum. Gene Ther. 10:641-648 (1999); Ali et al., Hum. Mol. Genet. 5:591-594 (1996); Samulski et al., J Vir. 63:3822-3828 (1989); Mendelson et al., Virol. 166:154-165 (1988); and Flotte et al., PNAS 90:10613-10617 (1993), which are hereby incorporated by reference in their entirety); SV40; herpes simplex virus; human immunodeficiency virus (see, e.g., Miyoshi et al., PNAS 94:10319-23 (1997); Takahashi et al., J. Virol. 73:781-7816 (1999), which are hereby incorporated by reference in their entirety); a retroviral vector, e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus and the like.

In some embodiments, the vector is a Tornado expression vector (see, e.g., Litke & Jaffrey, “Highly Efficient Expression of Circular RNA Aptamers in Cells Using Autocatalytic Transcripts,” Nat. Biotechnol. 37(6): 667-675(2019), which is hereby incorporated by reference in its entirety).

Nucleic acid sequences encoding the circular RNA molecules according to the present disclosure may be incorporated into a vector using standard cloning procedures in the art, as described by Maniatis et al., Molecular Cloning: A Laboratory Manual, Cold Springs Laboratory, Cold Springs Harbor, New York (1982), which is hereby incorporated by reference in its entirety.

### Host Cells

Once the DNA construct according to the present disclosure has been constructed, it can be incorporated into cells using conventional recombinant DNA technology. Accordingly, a further aspect of the disclosure relates to a host cell that includes a circular RNA molecule or a DNA construct according to the present disclosure.

In some embodiments, the host cell comprises an endogenous RNA ligase. In some embodiments, the endogenous RNA ligase has the ability to catalyze the circularization of a ribonucleic acid molecule having a 5′-OH and a 2′,3′-cyclic phosphate. In accordance with some embodiments, the endogenous RNA ligase is RtcB. It will be recognized that there are some enzymes that are related in function to RtcB, but not in sequence to RtcB. In some embodiments, the RNA ligase is any RNA ligase that detects 5′-OH and 2′-3′-cyclic phosphate ends.

The cell may be a eukaryotic cell. Exemplary eukaryotic cells include a yeast cell, an insect cell, a fungal cell, a plant cell, and an animal cell (e.g., a mammalian cell). Suitable mammalian cells include, for example without limitation, human, non-human primate, cat, dog, sheep, goat, cow, horse, pig, rabbit, and rodent cells. The host cell is preferably present either in a cell culture (ex vivo) or in a whole living organism (in vivo).

Suitable methods of introducing RNA molecules into cells are well known in the art and include, but are not limited to, the use of transfection reagents, electroporation, microinjection, or via viruses. Introducing the circular RNA molecules according to the present disclosure into a host cell can be carried out by the various forms of transformation, depending upon the vector/host cell system such as transformation, transduction, conjugation, mobilization, or electroporation.

### Pharmaceutical Compositions

Another aspect of the disclosure relates to a pharmaceutical composition comprising (i) a circular RNA molecule or a DNA construct according to the present disclosure and (ii) a pharmaceutically-acceptable carrier.

The pharmaceutical compositions may include a “pharmaceutically acceptable inert carrier,” and this expression is intended to include one or more inert excipients, which include, for example and without limitation, starches, polyols, granulating agents, microcrystalline cellulose, diluents, lubricants, binders, disintegrating agents, and the like. If desired, tablet dosages of the disclosed compositions may be coated by standard aqueous or nonaqueous techniques. “Pharmaceutically acceptable carrier” also encompasses controlled release means.

Pharmaceutical compositions may also optionally include other therapeutic ingredients, anti-caking agents, preservatives, sweetening agents, colorants, flavors, desiccants, plasticizers, dyes, and the like. Any such optional ingredient must be compatible with a circular RNA molecule as disclosed herein or a DNA construct as disclosed herein to insure the stability of the formulation. The composition may contain other additives as needed including, for example, lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example, alanine, glycine, and betaine, and peptides and proteins, for example, albumen.

Examples of excipients for use as the pharmaceutically acceptable carriers and the pharmaceutically acceptable inert carriers and the aforementioned additional ingredients include, but are not limited to, binders, fillers, disintegrants, lubricants, anti-microbial agents, and coating agents.

Pharmaceutical compositions provided by the present disclosure include compositions wherein the circular RNA molecule(s) or a DNA construct(s) according to the present disclosure is contained in a therapeutically effective amount, i.e., in an amount effective to achieve its intended purpose. The actual amount effective for a particular application will depend, inter alia, on the disease, condition, or disorder being treated. When administered in methods to treat a disease, condition, or disorder, such compositions will contain an amount of the circular RNA molecule as disclosed herein or a DNA construct effective to achieve the desired result, e.g., modulating the activity of a target molecule (e.g., an mRNA comprising an IRE), and/or reducing, eliminating, or slowing the progression of a symptoms (e.g., symptoms of iron overload). Determination of a therapeutically effective amount of circular RNA molecule as disclosed herein or a DNA construct is well within the capabilities of those skilled in the art, especially in light of the detailed disclosure herein.

### Methods of Treating a Disease or Disorder

A further aspect of the disclosure relates to a method of treating a disease or disorder mediated by iron overload. This method involves administering to a subject in need of treatment for a disease or disorder mediated by iron overload a circular RNA molecule, a DNA construct, or a pharmaceutical composition according to the present disclosure, where said administering is effective to treat the disease or disorder mediated by iron overload in the subject.

The terms “treat”, “treating”, “treatment”, and grammatical variations thereof mean subjecting an individual subject to a protocol, regimen, process, or remedy, in which it is desired to obtain a physiologic response or outcome in that subject, e.g., a patient. In particular, the methods and compositions of the present disclosure may be used to slow the development of disease symptoms or delay the onset of the disease or condition, or halt the progression of disease development. However, because every treated subject may not respond to a particular treatment protocol, regimen, process, or remedy, treating does not require that the desired physiologic response or outcome be achieved in each and every subject or subject, e.g., patient, population. Accordingly, a given subject or subject, e.g., patient, population may fail to respond or respond inadequately to treatment.

The term “subject” refers to any mammalian subject for whom diagnosis, treatment, or therapy is desired, particularly humans.

In some embodiments, the subject is a mammalian subject. The terms “mammal” or “mammalian subject” for purposes of the methods described herein refers to any animal classified as a mammal, including humans, domestic and farm animals, and zoo, sports, or pet animals, such as dogs, horses, cats, cows, sheep, goats, pigs, camels, etc.

In some embodiments, the mammalian subject is a human subject. The human subject may be an infant, a child, an adolescent, an adult, or a geriatric subject.

In some embodiments, the methods of the present disclosure find use in experimental animals, in veterinary application, and in the development of animal models, including, but not limited to, rodents including mice, rats, hamsters, and primates.

Subjects suitable for treatment in accordance with the methods described herein will vary and may include but are not limited to e.g., subjects suspected of having a disease or condition mediated by iron overload. The term “iron overload” refers to a condition in which excess iron is stored in the body. Excess iron may be deposited in organs throughout the body including, e.g., the liver, heart, and endocrine glands.

In some embodiments, the subject has a primary hemochromatosis, e.g., hereditary hemochromatosis or juvenile hemochromatosis. In primary hemochromatosis, increased intestinal iron absorption leads to excessive accumulations of iron, throughout the body, particularly in parenchymal cells (see, e.g., McLaren et al., “Iron Overload Disorders: Natural History, Pathogenesis, Diagnosis, and Therapy,” Crit. Rev. Clin. Lab Sci. 19(3):205-255 (1983), which is hereby incorporated by reference in its entirety).

In some embodiments, the subject has a secondary hemochromatosis, e.g., transfusional hemosiderosis, alcoholic cirrhosis, thalassemia, sideroblastic anemia, and porphyria cutanea tarda. In secondary hemochromatosis, iron accumulates in the reticuloendothelial system initially, but with increasing amounts of total body iron, excessive iron deposits eventually accumulate in parenchymal cells throughout the body producing a picture indistinguishable from hereditary hemochromatosis (see, e.g., McLaren et al., “Iron Overload Disorders: Natural History, Pathogenesis, Diagnosis, and Therapy,” Crit. Rev. Clin. Lab Sci. 19(3):205-255 (1983), which is hereby incorporated by reference in its entirety).

Iron overload may be manifested in subjects having hemochromatosis by, e.g., melanoderma, diabetes mellitus, and liver cirrhosis (McLaren et al., “Iron Overload Disorders: Natural History, Pathogenesis, Diagnosis, and Therapy,” Crit. Rev. Clin. Lab Sci. 19(3):205-255 (1983), which is hereby incorporated by reference in its entirety).

In some instances, subjects suitable for treatment in accordance with the methods described herein include subjects that do not have a disease or disorder mediated by iron overload but will be subjected to or otherwise exposed to conditions predicted to cause a disease or disorder mediated by iron overload. As such, in some instances, the methods described herein include preventing a disease or condition mediated by iron overload in a subject that does not have a disease or condition mediated by iron overload but is expected to be exposed to conditions that may cause a disease or disorder mediated by iron overload.

In some embodiments, the subject has a disease or disorder associated with ferroptosis. The term “ferroptosis” refers to an iron-dependent programmed cell death pathway driven by iron-dependent phospholipid peroxidation, which is regulated by multiple cellular metabolic pathways, including redox homeostasis, iron handling, mitochondrial activity and metabolism of amino acids, lipids and sugars, in addition to various signaling pathways relevant to disease (see, e.g., Jiang et al., “Ferroptosis: Mechanisms, Biology, and Role in Disease,” Nature 22: 266-282 (2021), which is hereby incorporated by reference in its entirety).

In some embodiments, the disease or disorder is selected from the group consisting of acute kidney injury, cancer, cardiovascular disease, neurodegenerative disease, and hepatic disease (see, e.g., Han et al., “Ferroptosis and Its Potential Role in Human Diseases,” Front. Pharmacol. 11: 239 (2020) and Qiu et al., The Application of Ferroptosis in Diseases,” Pharmacol. Res. 159: 104919 (2020), which are hereby incorporated by reference in their entirety).

As described herein supra, several diseases associated with elevated iron include: various neurodegenerative diseases, such as Alzheimer's, Huntington's, and Parkinson's diseases, Friedreich's ataxia, traumatic brain injury, and ischemic stroke. Thus, in some embodiments, the disease or disorder is a neurodegenerative disease. As used herein, the term “neurodegenerative disease” refers to a disease or condition in which the function of a subject's nervous system becomes impaired.

Accordingly, subjects selected for the methods described herein include those already afflicted with a neurodegenerative disease, as well as those at risk of having a neurodegenerative disease (i.e., in which prevention is desired). Such subjects include those with increased susceptibility to CNS injury, neurodegeneration, or neuroinflammation; those suspected of having CNS injury, neurodegeneration, or neuroinflammation; those with an increased risk of developing CNS injury, neurodegeneration, or neuroinflammation; those with increased environmental exposure to practices or agents causing CNS injury, neurodegeneration, or neuroinflammation, those suspected of having a genetic or behavioral predisposition to CNS injury, neurodegeneration, or neuroinflammation; those with CNS injury, neurodegeneration, or neuroinflammation, those having results from screening indicating an increased risk of CNS injury, neurodegeneration, or neuroinflammation, those having tested positive for a CNS injury, neurodegeneration, or neuroinflammation related condition; those having tested positive for one or more biomarkers of a CNS injury, neurodegeneration, or neuroinflammation related condition, etc.

Exemplary neurodegenerative diseases which subjects may have or be at risk of having for the purposes of the methods described herein include, without limitation, Alzheimer's disease, Parkinson's disease, Huntington's disease, multiple sclerosis, amyotrophic lateral sclerosis, prion disease, motor neuron diseases (MND), spinocerebellar ataxia (SCA), spinal muscular atrophy (SMA), eye-related neurodegenerative disease, e.g., glaucoma, diabetic retinopathy, age-related macular degeneration (AMD), and the like.

Additionally, transfusional iron overload and related conditions such as thalassemia major may be ameliorated with this technology. In some embodiments, the disease or disorder is a condition related to transfusional iron overload.

The term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The circular RNA molecule(s), DNA construct(s), or pharmaceutical composition(s) of the disclosure can be administered alone or can be co-administered to a subject. Co-administration is meant to include simultaneous or sequential administration of the circular RNA molecule(s), DNA construct(s), or pharmaceutical composition(s) of the disclosure individually or in combination (more than one compound or agent). Thus, the circular RNA molecule(s), DNA construct(s), or pharmaceutical composition(s) of the disclosure can also be combined, when desired, with other active substances (e.g., to reduce metabolic degradation). The circular RNA molecule(s), DNA construct(s), or pharmaceutical composition(s) of the disclosure can be formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The circular RNA molecule(s), DNA construct(s), or pharmaceutical composition(s) of the disclosure of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760, which are hereby incorporated by reference in their entirety. The circular RNA molecule(s), DNA construct(s), or pharmaceutical composition(s) of the disclosure can also be delivered as microspheres for slow release in the body. For example, microspheres can be administered via intradermal injection of drug-containing microspheres, which slowly release subcutaneously (see, e.g., Rao, J. Biomater Sci. Polym. Ed. 7:623-645 (1995), which is hereby incorporated by reference in its entirety; as biodegradable and injectable gel formulations (see, e.g., Gao Pharm. Res. 12:857-863 (1995), which is hereby incorporated by reference in its entirety); or, as microspheres for oral administration (see, e.g., Eyles, J. Pharm. Pharmacol. 49:669674, 1997, which is hereby incorporated by reference in its entirety).

In some embodiments, the circular RNA molecule(s), DNA construct(s), or pharmaceutical composition(s) of the disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo (see, e.g., Al-Muhammed, J. Microencapsul. 13:293-306 (1996); Chonn, Curr. Opin. Biotechnol. 6:698-708 (1995); and Ostro, Am. J. Hosp. Pharm. 46:1576-1587 (1989), which are hereby incorporated by reference in their entirety). The compositions of the present disclosure can also be delivered as nanoparticles.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cardiomyopathy or neurodegeneration such as Parkinson's disease and severity of such symptoms), kind of concurrent treatment, complications from the disease being treated or other health-related problems. Other therapeutic regimens or agents can be used in conjunction with the methods and compounds of the present disclosure. Adjustment and manipulation of established dosages (e.g., frequency and duration) are well within the ability of those skilled in the art.

For any circular RNA molecule as disclosed herein or a DNA construct described herein, the therapeutically effective amount can be initially determined from cell culture assays. Target concentrations will be those concentrations of active compound(s) that are capable of achieving the methods described herein, as measured using the methods described herein or known in the art.

Therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the subject and the circular RNA molecule as disclosed herein or a DNA construct being employed. The dose administered to a subject, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Various embodiments of the RNA molecules, DNA constructs, and pharmaceutical composition of the present disclosure are described above and apply in carrying out this treatment method. For example, in some embodiments, the DNA construct comprises the sequence of SEQ ID NO:2.

### EXAMPLES

The following examples are intended to exemplify the practice of embodiments of the disclosure but are by no means intended to limit the scope thereof.

### Example 1—Effects of Circular IRE RNA on Iron Metabolism in Cells

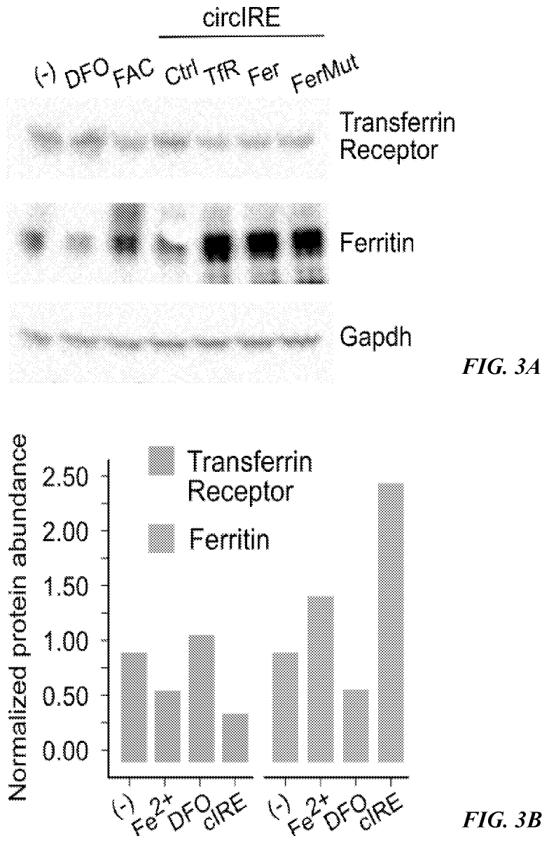

To determine the effect of circular TRE RNA on iron metabolism in cells, the level of transferrin receptor (iron import) and of ferritin (iron storage) was evaluated in different conditions by Western blot (FIGS. 3A-3B). Relative to untreated cells, desferoxamine (DFO) which chelates iron, led to a counterproductive compensatory increase in iron import and reduction in iron storage (FIGS. 3A-3B). Adding physiologically accessible iron to cells (ferric ammonic citrate, or FAC) reduced iron import and increased iron storage (FIGS. 3A-3B). A control circular RNA had minimal effects relative to untreated cells, while each of the circular TRE RNA (lane 5 (TfR; SEQ ID NO:3); lane 6 (Fer; SEQ ID NO:5); and lane 7 (FerMut)) led to a major increase in iron storage and a reduction of import (FIGS. 3A-3B).

### Example 2—Effects of Circular IRE RNA on Induction of Ferroptosis

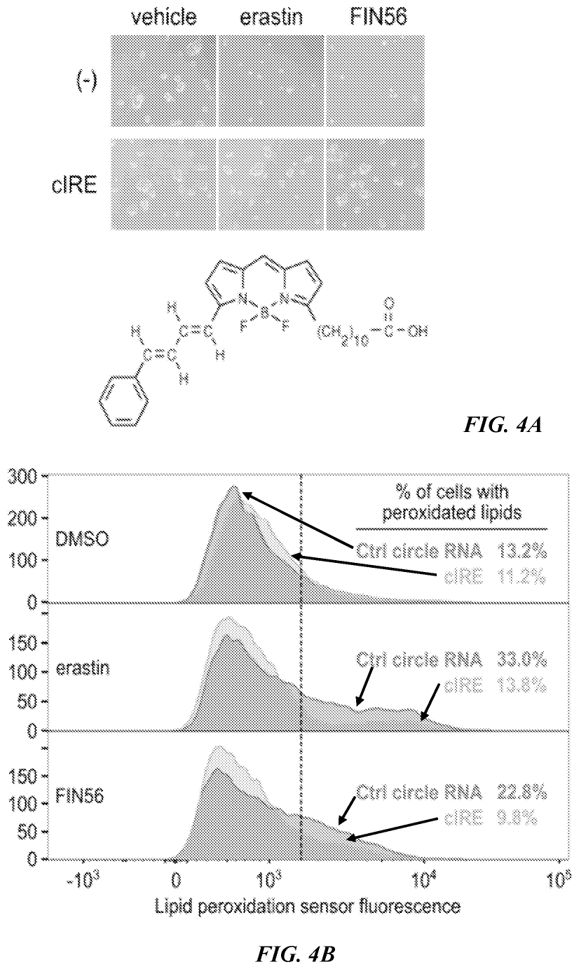

To determine the effect of circular TRE RNA on induction of ferroptosis, HepG2 cell viability was evaluated in untransfected cells and cells transfected with circular IREs (FIG. 4A). Following treatment with two ferroptosis inducers (erastin and FIN56), IRE expressing HepG2 cells remained viable, while the untransfected cells were nonviable (FIG. 4A).

Lipid peroxidation, a hallmark of ferroptosis, was evaluated in untransfected HEK293T cells and HEK293T cells transfected with circular IREs (FIG. 4B). Following treatment with two ferroptosis inducers (erastin and FIN56), lipid peroxidation was measured by increase of green fluorescence in pools of cells stained with an oxidizable BODIPY-C11 lipid conjugate. FIG. 4B demonstrates that ferroptosis is almost completely blocked in cells expressing circular IREs, while the percent of cells expressing the control RNA with oxidized lipids increased at most by over 2-fold.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A circular RNA molecule comprising:
an iron responsive element comprising the sequence of SEQ ID NO:1, SEQ ID NO: 4, or a portion of SEQ ID NO: 1 or SEQ ID NO: 4 sufficient to allow for binding to an iron responsive protein.

2. (canceled)

3. The circular RNA molecule of claim 1, wherein the iron responsive protein is iron regulatory protein 1 (“IRP1”) and/or iron regulatory protein 2 (“IRP2”).

4. The circular RNA molecule of claim 1, wherein the circular RNA molecule comprises a non-natural or modified nucleotide.

5. The circular RNA molecule of claim 1, said circular RNA molecule further comprising:
an RNA scaffold.

6. The circular RNA molecule of claim 1, said circular RNA molecule further comprising:
a fluorogenic aptamer.

7. The circular RNA molecule of claim 6, wherein the fluorogenic aptamer is selected from Squash, Beetroot, Spinach, Spinach 2, Broccoli, Red-Broccoli, Orange Broccoli, Corn, Mango, Malachite Green, cobalamine-binding aptamer, and derivatives thereof.

8. The circular RNA molecule of claim 7, wherein the fluorogenic aptamer binds to a fluorophore whose fluorescence, absorbance, spectral properties, or quenching properties are increased, decreased, or altered by interaction with the fluorogenic aptamer.

9. A DNA construct encoding the circular RNA molecule according to claim 1.

10. The DNA construct of claim 9, wherein said DNA construct comprises a nucleic acid sequence encoding:
(i) a first self-cleaving ribozyme;
(ii) a first ligation sequence;
(iii) the iron responsive element comprising the sequence of SEQ ID NO:1 or SEQ ID NO:4;
(iv) a second ligation sequence; and
(v) a second self-cleaving ribozyme.

11. The DNA construct of claim 10, wherein said DNA construct further comprises:
a promoter operatively coupled to the nucleic acid sequence.

12. The DNA construct of claim 11, wherein the promoter is a prokaryotic promoter selected from the group consisting of T7, T3, SP6 RNA polymerases, and derivatives thereof.

13. The DNA construct of claim 11, wherein the promoter is:
(i) a eukaryotic RNA Polymerase II promoter or a derivative thereof or
(ii) a eukaryotic RNA polymerase I promoter or RNA Polymerase III promoter selected from the group consisting of U6, H1, 5S, 7SK promoter, and derivatives thereof.

14. (canceled)

15. The DNA construct according to claim 9, wherein the DNA construct comprises the sequence of SEQ ID NO:2.

16. The DNA construct according to claim 9, wherein the DNA construct is an expression vector.

17. A cell comprising the circular RNA molecule of claim 1.

18-20. (canceled)

21. A pharmaceutical composition comprising:
(i) the circular RNA molecule according to claim 1 and
(ii) a pharmaceutically-acceptable carrier.

22. A method of treating a disease or disorder mediated by iron overload, the method comprising:
administering, to a subject in need of treatment for a disease or disorder mediated by iron overload, a circular RNA molecule according to claim 1, wherein said administering is effective to treat the disease or disorder mediated by iron overload in the subject.

23. The method of claim 22, wherein the subject is a mammalian subject.

24. (canceled)

25. The method according to claim 22, wherein the subject has a primary or secondary hemochromatosis.

26. The method according to claim 22, wherein the subject has a disease or disorder associated with ferroptosis.

27. (canceled)


SEQUENCE LISTING (5 sequences)

SEQ ID NO: 1 - RNA, 26 nt, synthetic construct
  source 1..26: mol_type = other RNA
  misc_feature 1..26: note = iron responsive element consensus sequence
nnntgcnnnnncwrtghnnnnncnnn

SEQ ID NO: 2 - DNA, 326 bp, synthetic construct
  source 1..326: mol_type = other DNA
  misc_feature 1..326: note = circular iron responsive element construct
  misc_feature 11..71: note = ribozyme 1
  misc_feature 79..90: note = F30 scaffold
  misc_feature 176..188: note = F30 scaffold
  misc_feature 232..244: note = F30 scaffold
  misc_feature 98..168: note = Squash
  misc_feature 196..224: note = Iron responsive element
  misc_feature 261..314: note = ribozyme 2
ggccgcactcgccggtcccaagcccggataaaatgggagggggcgggaaaccgcctaaccatgccgagtgcggccgcttgccatgtgtatcggtccgctacaaggtgagcccaataatacggtttgggttaggataggaagtagagccgtaaactctctaagcggtagcggtccgatactctgatgatgggtcccgattatcgggagcagtgtcttccataatcgggtcccatcattcatggcaagtggccgcggtcggcgtggactgtagaacactgccaatgccggtcccaagcccggataaaagtggagggtacagtccacgc

SEQ ID NO: 3 - RNA, 23 nt, synthetic construct
  source 1..23: mol_type = other RNA
  misc_feature 1..23: note = iron responsive element
tatcgggagcagtgtcttccata

SEQ ID NO: 4 - RNA, 26 nt, synthetic construct
  source 1..26: mol_type = other RNA
  misc_feature 1..26: note = iron responsive element consensus sequece
nnntgcnnnnncwrtghnnnnncnnn

SEQ ID NO: 5 - RNA, 26 nt, synthetic construct
  source 1..26: mol_type = other RNA
  misc_feature 1..26: note = iron responsive element
tcttgcttcaacagtgtttgaacgga